(19) United States
(12) Patent Application Publication
(10) Pub. No.: US 2021/0187483 A1
(43) Pub. Date: Jun. 24, 2021

(54) CATALYST AND METHOD OF HYDROGENATING UNSATURATED COMPOUND WITH MULTI-CARBOXYLIC ACID GROUPS

(71) Applicants: INDUSTRIAL TECHNOLOGY RESEARCH INSTITUTE, Hsinchu (TW); CHUNG HWA CHEMICAL INDUSTRIAL WORKS, LTD., Taoyuan City (TW)

(72) Inventors: Shu-Hua CHAN, Taichung City (TW); Hsi-Yen HSU, Hsinchu City (TW); Chien-Wei HSU, Houlong Township (TW); Yen-Horng TSOU, New Taipei City (TW); Shi-Hung HSU, Taoyuan City (TW)

(73) Assignees: INDUSTRIAL TECHNOLOGY RESEARCH INSTITUTE, Hsinchu (TW); CHUNG HWA CHEMICAL INDUSTRIAL WORKS, LTD., Taoyuan City (TW)

(21) Appl. No.: 16/720,981

(22) Filed: Dec. 19, 2019

(51) Int. Cl. B01J 23/54; C07C 51/36

(52) U.S. Cl. CPC B01J 23/54; B01J 21/04; C07C 51/36

(57) ABSTRACT

A method of hydrogenating unsaturated compound with multi-carboxylic acid groups is provided, which includes introducing hydrogen to an unsaturated compound with multi-carboxylic acid groups in the presence of a catalyst to hydrogenate the alkene or alkyne group of the unsaturated compound with multi-carboxylic acid groups without hydrogenating the carboxylic acid groups of the unsaturated compound with multi-carboxylic acid groups. The catalyst includes a support, and palladium and metal oxide loaded on the support.

### TECHNICAL FIELD

The technical field relates to a method of hydrogenating unsaturated compound with multi-carboxylic acid groups, and in particular it relates to a catalyst utilized by the method.

### BACKGROUND

Itaconic acid is white powder crystal, which can be stably stored, and the crystal product does not absorb moisture. The molecular structure of the itaconic acid has an unsaturated double bond and two carboxylic acid groups (belonging to groups of high reactivity), which is suitable for developing various derivatives and intermediate raw materials. The itaconic acid can be hydrogenated to form various itaconic derivatives. 2-methylsuccinic acid is one of the hydrogenated derivatives of itaconic acid, which is white crystal and easily dissolved in water, ethanol, or tetrahydrofuran. 2-Methylsuccinic acid is widely applied in several fields, which is generally used in synthesizing polyurethane monomer, etchant, esterification, or photochemical reaction. Moreover, 2-methylsuccinic acid is a necessary raw material to synthesize macromolecular drugs. However, the traditional method of synthesizing 2-methylsuccinic acid faces lots of issues such as low yield, complicated process of preparation and purification, and too much waste liquid.

Accordingly, a novel catalyst is called for in directly hydrogenation of itaconic acid to form 2-methylsuccinic acid at a lower reaction temperature and pressure, for a shorter reaction time while maintaining a high product yield.

### SUMMARY

One embodiment of the disclosure provides a catalyst for hydrogenating unsaturated compound with multi-carboxylic acid groups, including: a support; palladium and metal oxide loaded on the support, wherein the metal oxide is zinc oxide, cerium oxide, zirconium oxide, or a combination thereof.

In some embodiments, the support includes aluminum oxide or silicon dioxide.

In some embodiments, the support and the palladium have a weight ratio of 100:0.3 to 100:3.

In some embodiments, the support and the metal oxide have a weight ratio of 100:1 to 100:5.

In some embodiments, the unsaturated compound with multi-carboxylic acid groups includes itaconic acid, maleic acid, maleic anhydride, fumaric acid, methyl maleic acid, methyl fumaric acid, tetrahydrophthalic acid, tetrahydrophthalic anhydride, methyl tetrahydrophthalic anhydride, or glutaric acid.

One embodiment of the disclosure provides a method of hydrogenating unsaturated compound with multi-carboxylic acid groups, including introducing hydrogen into an unsaturated compound with multi-carboxylic acid groups in the presence of a catalyst to hydrogenate an alkene or alkyne group of the unsaturated compound with multi-carboxylic acid groups but without hydrogenating carboxylic acid groups of the unsaturated compound with multi-carboxylic acid groups, wherein the catalyst includes: a support; and palladium and metal oxide loaded on the support, wherein the metal oxide is zinc oxide, cerium oxide, zirconium oxide, or a combination thereof.

In some embodiments, the hydrogen gas has a pressure of 3 bar to 50 bar.

In some embodiments, the step of hydrogenating the alkene or alkyne group of the unsaturated compound with multi-carboxylic acid groups is performed at a temperature of 30° C. to 100° C.

In some embodiments, the support includes aluminum oxide or silicon dioxide.

In some embodiments, the support and the palladium have a weight ratio of 100:0.3 to 100:3.

In some embodiments, the support and the metal oxide have a weight ratio of 100:1 to 100:5.

In some embodiments, the unsaturated compound with multi-carboxylic acid groups includes itaconic acid, maleic acid, maleic anhydride, fumaric acid, methyl maleic acid, methyl fumaric acid, tetrahydrophthalic acid, tetrahydrophthalic anhydride, methyl tetrahydrophthalic anhydride, or glutaric acid.

In some embodiments, the method is performed in a batchwise reactor or a continuous reactor.

### DETAILED DESCRIPTION

In the following detailed description, for purposes of explanation, numerous specific details are set forth in order to provide a thorough understanding of the disclosed embodiments. It will be apparent, however, that one or more embodiments may be practiced without these specific details.

One embodiment of the disclosure provides a catalyst for hydrogenating unsaturated compound with multi-carboxylic acid groups, including a support, and palladium and metal oxide loaded on the support. For example, the metal oxide includes zinc oxide, cerium oxide, zirconium oxide, or a combination thereof. If the metal oxide is another oxide such as magnesium oxide or lanthanum oxide, the hydrogenation efficiency of hydrogenating unsaturated compound with multi-carboxylic acid groups will be greatly lowered. In some embodiments, the support includes aluminum oxide or silicon dioxide. For example, the support may have a diameter of 20 μm to 200 μm. If the support diameter is too small, it will not be easy to separate the catalyst from the product during purification. If the support diameter is too large, the surface area of the support will be less and the catalyst coverage amount will be decreased. In some embodiments, the support can be porous material with a pore size of 2 nm to 100 nm. Pores may increase the surface area of the support so to increase the catalyst coverage amount per unit weight of the support. If the pore size of the porous support is too small, the catalyst coverage amount per unit weight of the porous catalyst will be similar to non-porous catalyst.

In some embodiments, the support and the palladium have a weight ratio of 100:0.3 to 100:3. If the palladium ratio is too low, the hydrogenation efficiency will be poor. If the palladium ratio is too high, the catalyst cost will be increased. In some embodiments, the support and the metal oxide have a weight ratio of 100:1 to 100:5. If the metal oxide ratio is too low, the hydrogenation efficiency will be poor. In one embodiment, the metal oxide amount is higher than the palladium amount.

In some embodiments, the unsaturated compound with multi-carboxylic acid groups is a compound having double bond (alkene group) or triple bond (alkyne group) and multi carboxylic acid groups. For example, the unsaturated compound with multi-carboxylic acid groups can be itaconic acid, maleic acid, maleic anhydride, fumaric acid, methyl maleic acid, methyl fumaric acid, tetrahydrophthalic acid, tetrahydrophthalic anhydride, methyl tetrahydrophthalic anhydride, glutaric acid, or another suitable unsaturated compound with multi-carboxylic acid groups. In one embodiment, the unsaturated compound with multi-carboxylic acid groups is itaconic acid.

In one embodiment, the method of hydrogenating unsaturated compound with multi-carboxylic acid groups includes introducing hydrogen into an unsaturated compound with multi-carboxylic acid groups in the presence of a catalyst, thereby hydrogenating an alkene or alkyne group of the unsaturated compound with multi-carboxylic acid groups but without hydrogenating carboxylic acid groups of the unsaturated compound with multi-carboxylic acid groups. When the unsaturated compound with multi-carboxylic acid groups is itaconic acid, the hydrogenation scheme is shown below:

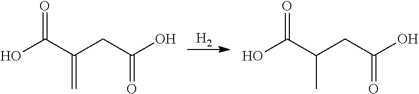

Obviously, the above mentioned hydrogenation reaction only hydrogenates the double bond of the itaconic acid without hydrogenating the two carboxylic acid groups. The unsaturated compound with multi-carboxylic acid groups is placed in a reactor to perform hydrogenation. The reactor can be a continuous type (e.g. trickle bed reactor) or a non-continuous type (e.g. batchwise reactor). In some embodiments, the hydrogen pressure is between 3 bar to 50 bar. If the hydrogen pressure is too low, the reaction rate and the conversion ratio of the hydrogenation will be lowered. If the hydrogen pressure is too high, the operation will be dangerous. In some embodiments, the hydrogenation reaction of the alkene or alkyne group of the unsaturated compound with multi-carboxylic acid groups is performed at a temperature of 30° C. to 100° C. If the hydrogenation temperature is too low, the reaction rate and the conversion ratio of the hydrogenation will be lowered. If the hydrogenation temperature is too high, the hydrogenation selectivity will be lowered and side products will be increased.

Below, exemplary embodiments will be described in detail so as to be easily realized by a person having ordinary knowledge in the art. The inventive concept may be embodied in various forms without being limited to the exemplary embodiments set forth herein. Descriptions of well-known parts are omitted for clarity, and like reference numerals refer to like elements throughout.

### EXAMPLES

### Example 1 (1 wt % Pd/4 wt % ZnO/Al₂O₃)

95 g of palladium chloride solution (having a Pd concentration of 5 wt %), 78 g of zinc nitrate, and 288 g of water were mixed to form a uniform metal salt solution. 500 g of dried aluminum oxide powder was placed into a mixing tank, the metal salt solution was then slowly dropwise added into the mixing tank, and the stirring blades of the mixer were switched on at same time. The metal salt solution was all added after stirring for about 20 to 30 minutes, and the stirring was then continued for 5 minutes. After stopping stirring, the impregnated aluminum oxide was placed onto a ceramic plate, dried by baking at 150° C. for 4 hours, and then sintered at 450° C. for 4 hours. The sintered catalyst was cooled at room temperature. The catalyst powder was added to 1 kg of water and stirred for 10 minutes, and the solid part was collected by filtering. The filtered catalyst was repeatedly washed with water 3 to 5 times, and then dried by baking at 150° C. for 4 hours to obtain catalyst powder (1 wt % Pd/4 wt % ZnO/Al₂O₃). The catalyst powder was chemically reduced under hydrogen atmosphere at 250° C. for 4 hours before serving as a hydrogenation catalyst.

0.5 g of the above catalyst was placed into an autoclave, and 30 g of itaconic acid and 170 g of de-ionized water were then added into the autoclave. The mixture in the autoclave was heated to 50° C., and pressure of hydrogen that was introduced into the autoclave was adjusted to 150 psi to perform hydrogenation for 60 minutes. The hydrogenation product was analyzed by high performance liquid chromatography (HPLC), in which the itaconic acid was hydrogenated to 2-methylsuccinic acid in a conversion ratio of 99.8%.

### Example 2 (1 wt % Pd/4 wt % CeO₂/Al₂O₃)

95 g of palladium chloride solution (having a Pd concentration of 5 wt %), 51 g of cerium nitrate, and 315 g of water were mixed to form a uniform metal salt solution. 500 g of dried aluminum oxide powder was placed into a mixing tank, the metal salt solution was then slowly dropwise added into the mixing tank, and the stirring blades of the mixer were switched on at same time. The metal salt solution was all added after stirring for about 20 to 30 minutes, and the stirring was then continued for 5 minutes. After stopping stirring, the impregnated aluminum oxide was placed onto a ceramic plate, dried by baking at 150° C. for 4 hours, and then sintered at 450° C. for 4 hours. The sintered catalyst was cooled at room temperature. The catalyst powder was added to 1 kg of water and stirred for 10 minutes, and the solid part was collected by filtering. The filtered catalyst was repeatedly washed with water 3 to 5 times, and then dried by baking at 150° C. for 4 hours to obtain catalyst powder (1 wt % Pd/4 wt % CeO₂/Al₂O₃). The catalyst powder was chemically reduced under hydrogen atmosphere at 250° C. for 4 hours before serving as a hydrogenation catalyst.

0.5 g of the above catalyst was placed into an autoclave, and 30 g of itaconic acid and 170 g of de-ionized water were then added into the autoclave. The mixture in the autoclave was heated to 50° C., and pressure of hydrogen that was introduced into the autoclave was adjusted to 150 psi to perform hydrogenation for 60 minutes. The hydrogenation product was analyzed by HPLC, in which the itaconic acid was hydrogenated to 2-methylsuccinic acid in a conversion ratio of 99.9%.

### Example 3 (1 wt % Pd/4 wt % ZrO₂/Al₂O₃)

95 g of palladium chloride solution (having a Pd concentration of 5 wt %), 44 g of zirconium oxynitrate, and 325 g of water were mixed to form a uniform metal salt solution. 500 g of dried aluminum oxide powder was placed into a mixing tank, the metal salt solution was then slowly dropwise added into the mixing tank, and the stirring blades of the mixer were switched on at same time. The metal salt solution was all added after stirring for about 20 to 30 minutes, and the stirring was then continued for 5 minutes. After stopping stirring, the impregnated aluminum oxide was placed onto a ceramic plate, dried by baking at 150° C. for 4 hours, and then sintered at 450° C. for 4 hours. The sintered catalyst was cooled at room temperature. The catalyst powder was added to 1 kg of water and stirred for 10 minutes, and the solid part was collected by filtering. The filtered catalyst was repeatedly washed with water 3 to 5 times, and then dried by baking at 150° C. for 4 hours to obtain catalyst powder (1 wt % Pd/4 wt % ZrO₂/Al₂O₃). The catalyst powder was chemically reduced under hydrogen atmosphere at 250° C. for 4 hours before serving as a hydrogenation catalyst.

0.5 g of the above catalyst was placed into an autoclave, and 30 g of itaconic acid and 170 g of de-ionized water were then added into the autoclave. The mixture in the autoclave was heated to 50° C., and pressure of hydrogen that was introduced into the autoclave was adjusted to 150 psi to perform hydrogenation for 60 minutes. The hydrogenation product was analyzed by HPLC, in which the itaconic acid was hydrogenated to 2-methylsuccinic acid in a conversion ratio of 98.8%.

### Example 4 (1 wt % Pd/2 wt % CeO₂/Al₂O₃)

95 g of palladium chloride solution (having a Pd concentration of 5 wt %), 26 g of cerium nitrate, and 315 g of water were mixed to form a uniform metal salt solution. 500 g of dried aluminum oxide powder was placed into a mixing tank, the metal salt solution was then slowly dropwise added into the mixing tank, and the stirring blades of the mixer were switched on at same time. The metal salt solution was all added after stirring for about 20 to 30 minutes, and the stirring was then continued for 5 minutes. After stopping stirring, the impregnated aluminum oxide was placed onto a ceramic plate, dried by baking at 150° C. for 4 hours, and then sintered at 450° C. for 4 hours. The sintered catalyst was cooled at room temperature. The catalyst powder was added to 1 kg of water and stirred for 10 minutes, and the solid part was collected by filtering. The filtered catalyst was repeatedly washed with water 3 to 5 times, and then dried by baking at 150° C. for 4 hours to obtain catalyst powder (1 wt % Pd/2 wt % CeO₂/Al₂O₃). The catalyst powder was chemically reduced under hydrogen atmosphere at 250° C. for 4 hours before serving as a hydrogenation catalyst.

0.5 g of the above catalyst was placed into an autoclave, and 30 g of itaconic acid and 70 g of methanol were then added into the autoclave. The mixture in the autoclave was heated to 50° C., and pressure of hydrogen that was introduced into the autoclave was adjusted to 150 psi to perform hydrogenation for 60 minutes. The hydrogenation product was analyzed by HPLC, in which the itaconic acid was hydrogenated to 2-methylsuccinic acid in a conversion ratio of 100.0%.

### Comparative Example 1 (1 wt % Pd/4 wt % La₂O₃/Al₂O₃)

95 g of palladium chloride solution (having a Pd concentration of 5 wt %) and 775 g of water were mixed to form a uniform metal salt solution. 500 g of dried lanthanum-containing aluminum oxide powder (containing about 4 wt % of lanthanum) was placed into a mixing tank, the metal salt solution was then slowly dropwise added into the mixing tank, and the stirring blades of the mixer were switched on at same time. The metal salt solution was all added after stirring for about 20 to 30 minutes, and the stirring was then continued for 5 minutes. After stopping stirring, the impregnated lanthanum-containing aluminum oxide was placed onto a ceramic plate, dried by baking at 150° C. for 4 hours, and then sintered at 450° C. for 4 hours. The sintered catalyst was cooled at room temperature. The catalyst powder was added to 1 kg of water and stirred for 10 minutes, and the solid part was collected by filtering. The filtered catalyst was repeatedly washed with water 3 to 5 times, and then dried by baking at 150° C. for 4 hours to obtain catalyst powder (1 wt % Pd/4 wt % La₂O₃/Al₂O₃). The catalyst powder was chemically reduced under hydrogen atmosphere at 250° C. for 4 hours before serving as a hydrogenation catalyst.

0.5 g of the above catalyst was placed into an autoclave, and 30 g of itaconic acid and 170 g of de-ionized water were then added into the autoclave. The mixture in the autoclave was heated to 50° C., and pressure of hydrogen that was introduced into the autoclave was adjusted to 150 psi to perform hydrogenation for 60 minutes. The hydrogenation product was analyzed by HPLC, in which the itaconic acid was hydrogenated to 2-methylsuccinic acid in a conversion ratio of 4.8%.

### Comparative Example 2 (1 wt % Pd/9 wt % MgO/Al₂O₃)

95 g of palladium chloride solution (having a Pd concentration of 5 wt %) and 775 g of water were mixed to form a uniform metal salt solution. 500 g of dried magnesium-containing aluminum oxide powder (containing about 9 wt % of magnesium) was placed into a mixing tank, the metal salt solution was then slowly dropwise added into the mixing tank, and the stirring blades of the mixer were switched on at same time. The metal salt solution was all added after stirring for about 20 to 30 minutes, and the stirring was then continued for 5 minutes. After stopping stirring, the impregnated magnesium-containing aluminum oxide was placed onto a ceramic plate, dried by baking at 150° C. for 4 hours, and then sintered at 450° C. for 4 hours. The sintered catalyst was cooled at room temperature. The catalyst powder was added to 1 kg of water and stirred for 10 minutes, and the solid part was collected by filtering. The filtered catalyst was repeatedly washed with water 3 to 5 times, and then dried by baking at 150° C. for 4 hours to obtain catalyst powder (1 wt % Pd/9 wt % MgO/Al₂O₃). The catalyst powder was chemically reduced under hydrogen atmosphere at 250° C. for 4 hours before serving as a hydrogenation catalyst.

0.5 g of the above catalyst was placed into an autoclave, and 30 g of itaconic acid and 170 g of de-ionized water were then added into the autoclave. The mixture in the autoclave was heated to 50° C., and pressure of hydrogen that was introduced into the autoclave was adjusted to 150 psi to perform hydrogenation for 60 minutes. The hydrogenation product was analyzed by HPLC, in which the itaconic acid was hydrogenated to 2-methylsuccinic acid in a conversion ratio of 1.0%.

### Comparative Example 3 (1 wt % Pd/2 wt % Ru/Al₂O₃)

3.8 g of palladium chloride solution (having a Pd concentration of 5 wt %), 4.2 g of ruthenium chloride (having a Ru concentration of 9.4 wt %), and 10 g of water were mixed to form a uniform metal salt solution. 19 g of dried aluminum oxide powder was placed into a mixing tank, the metal salt solution was then slowly dropwise added into the mixing tank, and the stirring blades of the mixer were switched on at same time. The metal salt solution was all added after stirring for about 20 to 30 minutes, and the stirring was then continued for 5 minutes. After stopping stirring, the impregnated aluminum oxide was placed onto a ceramic plate, dried by baking at 150° C. for 4 hours, and then sintered at 450° C. for 4 hours. The sintered catalyst was cooled at room temperature. The catalyst powder was added to 1 kg of water and stirred for 10 minutes, and the solid part was collected by filtering. The filtered catalyst was repeatedly washed with water 3 to 5 times, and then dried by baking at 150° C. for 4 hours to obtain catalyst powder (1 wt % Pd/2 wt % Ru/Al₂O₃). The catalyst powder was chemically reduced under hydrogen atmosphere at 250° C. for 4 hours before serving as a hydrogenation catalyst.

0.5 g of the above catalyst was placed into an autoclave, and 30 g of itaconic acid and 170 g of de-ionized water were then added into the autoclave. The mixture in the autoclave was heated to 50° C., and pressure of hydrogen that was introduced into the autoclave was adjusted to 150 psi to perform hydrogenation for 60 minutes. The hydrogenation product was analyzed by HPLC, in which the itaconic acid was hydrogenated to 2-methylsuccinic acid in a conversion ratio of 99.3%. However, the ruthenium metal would leach out from the catalyst powder after the hydrogenation and could not be used again.

### Comparative Example 4 (10 wt % Ni/Al₂O₃)

9.7 g of nickel nitrate solution (having a Ni concentration of 10 wt %) and 6 g of water were mixed to form a uniform metal salt solution. 19.3 g of dried aluminum oxide powder was placed into a mixing tank, the metal salt solution was then slowly dropwise added into the mixing tank, and the stirring blades of the mixer were switched on at same time. The metal salt solution was all added after stirring for about 20 to 30 minutes, and the stirring was then continued for 5 minutes. After stopping stirring, the impregnated aluminum oxide was placed onto a ceramic plate, dried by baking at 150° C. for 4 hours, and then sintered at 450° C. for 4 hours. The sintered catalyst was cooled at room temperature. The catalyst powder was added to 1 kg of water and stirred for 10 minutes, and the solid part was collected by filtering. The filtered catalyst was repeatedly washed with water 3 to 5 times, and then dried by baking at 150° C. for 4 hours to obtain catalyst powder (10 wt % Ni/Al₂O₃). The catalyst powder was chemically reduced under hydrogen atmosphere at 250° C. for 4 hours before serving as a hydrogenation catalyst.

0.5 g of the above catalyst was placed into an autoclave, and 30 g of itaconic acid and 170 g of de-ionized water were then added into the autoclave. The mixture in the autoclave was heated to 50° C., and pressure of hydrogen that was introduced into the autoclave was adjusted to 150 psi to perform hydrogenation for 60 minutes. The hydrogenation product was analyzed by HPLC, in which the itaconic acid was hydrogenated to 2-methylsuccinic acid in a conversion ratio of 14.5%.

### Comparative Example 5 (1 wt % Pd/Al₂O₃, Commercially Available Catalyst)

0.5 g of 1 wt % Pd/Al₂O₃ catalyst (commercially available from Aldrich) was placed into an autoclave, and 30 g of itaconic acid and 170 g of de-ionized water were then added into the autoclave. The mixture in the autoclave was heated to 50° C., and pressure of hydrogen that was introduced into the autoclave was adjusted to 150 psi to perform hydrogenation for 60 minutes. The hydrogenation product was analyzed by HPLC, in which the itaconic acid was hydrogenated to 2-methylsuccinic acid in a conversion ratio of 9.1%.

TABLE 1

| | | | | Conversion | |
|---|---|---|---|---|---|
| | Metal | Metal oxide | Support | ratio | Note |
| Example 1 | 1 wt % Pd | 4% ZnO | Al₂O₃ | 99.8% | NA |
| Example 2 | 1 wt % Pd | 4% CeO₂ | Al₂O₃ | 99.9% | NA |
| Example 3 | 1 wt % Pd | 4% ZrO₂ | Al₂O₃ | 98.8% | NA |
| Example 4 | 1 wt % Pd | 2% CeO₂ | Al₂O₃ | 100.0% | NA |
| Comparative | 1 wt % Pd | 4% La₂O₃ | Al₂O₃ | 4.8% | NA |
| Example 1 | | | | | |
| Comparative | 1 wt % Pd | 9% MgO | Al₂O₃ | 1.0% | NA |
| Example 2 | | | | | |
| Comparative | 1 wt % Pd/2 | None | Al₂O3 | 99.3% | Ruthenium |
| Example 3 | wt % Ru | | | | metal - |
| | | | | | was leached |
| | | | | | out from |
| | | | | | the catalyst |
| | | | | | powder and |
| | | | | | could not |
| | | | | | be used |
| | | | | | again |
| Comparative | 10 wt % Ni | None | Al₂O₃ | 14.5% | NA |
| Example 4 | | | | | |
| Comparative | 1 wt % Pd | None | Al₂O₃ | 9.1% | NA |
| Example 5 | | | | | |

As shown above, not all metal oxides were suitable to be collocated with Pd to hydrogenate the itaconic acid. If Pd was collocated with zinc oxide, cerium oxide, and zirconium oxide, the hydrogenation could be completed in 1 hour. If Pd was collocated with lanthanum oxide or magnesium oxide, the hydrogenation rate would be obviously lowered. On the other hand, if no metal oxide was used, even the normal commercial hydrogenation catalyst such as Ni or Pd could not efficiently hydrogenate the itaconic acid. In addition, some catalyst combination could be used only one time. For example, when Pd was collocated with the ruthenium chloride to serve as catalyst, the Ru metal was easily leach out from the reaction and consequently change the solution color.

### Example 5

1 g of the catalyst in Example 4 (1 wt % Pd/2 wt % CeO₂/Al₂O₃) was placed into an autoclave, and 30 g of itaconic acid and 70 g of methanol were then added into the autoclave. The mixture in the autoclave was heated to 50° C., and pressure of hydrogen that was introduced into the autoclave was adjusted to 150 psi to perform hydrogenation for 40 minutes. The hydrogenation product taken out of the autoclave (and the catalyst was kept in the autoclave) was analyzed by HPLC. 30 g of itaconic acid and 70 g of methanol were then added into the autoclave. The mixture in the autoclave was heated to 50° C., and pressure of hydrogen that was introduced into the autoclave was adjusted to 150 psi to perform hydrogenation (2nd cycle) for 40 minutes. The hydrogenation product taken out of the autoclave (and the catalyst was kept in the autoclave) was analyzed by HPLC. The above cycle was repeated 5 times.

TABLE 2

| | | | Tem- | | | |
|---|---|---|---|---|---|---|
| Cycle | Itaconic | Methanol | perature | Pressure | Time | Conversion |
| (number) | acid (g) | (g) | (° C.) | (psi) | (min) | ratio (%) |
| 1 | 30 | 70 | 50 | 150 | 40 | >99 |
| 2 | 30 | 70 | 50 | 150 | 40 | >99 |
| 3 | 30 | 70 | 50 | 150 | 40 | >99 |
| 4 | 30 | 70 | 50 | 150 | 40 | >99 |
| 5 | 30 | 70 | 50 | 150 | 40 | >99 |

As shown in Table 2, the catalyst in Example still had an excellent hydrogenation effect after being used several times.

It will be apparent to those skilled in the art that various modifications and variations can be made to the disclosed methods and materials. It is intended that the specification and examples be considered as exemplary only, with the true scope of the disclosure being indicated by the following claims and their equivalents.

1. A catalyst for hydrogenating unsaturated compound with multi-carboxylic acid groups, comprising:
a support; and
palladium and metal oxide loaded on the support, wherein the metal oxide is zinc oxide, cerium oxide, zirconium oxide, or a combination thereof.

2. The catalyst for hydrogenating unsaturated compound with multi-carboxylic acid groups as claimed in claim 1, wherein the support comprises aluminum oxide or silicon dioxide.

3. The catalyst for hydrogenating unsaturated compound with multi-carboxylic acid groups as claimed in claim 1, wherein the support and the palladium have a weight ratio of 100:0.3 to 100:3.

4. The catalyst for hydrogenating unsaturated compound with multi-carboxylic acid groups as claimed in claim 1, wherein the support and the metal oxide have a weight ratio of 100:1 to 100:5.

5. The catalyst for hydrogenating unsaturated compound with multi-carboxylic acid groups as claimed in claim 1, wherein the unsaturated compound with multi-carboxylic acid groups comprises itaconic acid, maleic acid, maleic anhydride, fumaric acid, methyl maleic acid, methyl fumaric acid, tetrahydrophthalic acid, tetrahydrophthalic anhydride, methyl tetrahydrophthalic anhydride, or glutaric acid.

6. A method of hydrogenating unsaturated compound with multi-carboxylic acid groups, comprising:
introducing hydrogen into an unsaturated compound with multi-carboxylic acid groups in the presence of a catalyst to hydrogenate an alkene or alkyne group of the unsaturated compound with multi-carboxylic acid groups without hydrogenating carboxylic acid groups of the unsaturated compound with multi-carboxylic acid groups,
wherein the catalyst comprises:
a support; and
palladium and metal oxide loaded on the support, wherein the metal oxide is zinc oxide, cerium oxide, zirconium oxide, or a combination thereof.

7. The method of hydrogenating unsaturated compound with multi-carboxylic acid groups as claimed in claim 6, wherein the hydrogen has a pressure of 3 bar to 50 bar.

8. The method of hydrogenating unsaturated compound with multi-carboxylic acid groups as claimed in claim 6, wherein hydrogenation of the alkene or the alkyne group of the unsaturated compound with multi-carboxylic acid groups is performed at a temperature of 30° C. to 100° C.

9. The method of hydrogenating unsaturated compound with multi-carboxylic acid groups as claimed in claim 6, wherein the support comprises aluminum oxide or silicon dioxide.

10. The method of hydrogenating unsaturated compound with multi-carboxylic acid groups as claimed in claim 6, wherein the support and the palladium have a weight ratio of 100:0.3 to 100:3.

11. The method of hydrogenating unsaturated compound with multi-carboxylic acid groups as claimed in claim 6, wherein the support and the metal oxide have a weight ratio of 100:1 to 100:5.

12. The method of hydrogenating unsaturated compound with multi-carboxylic acid groups as claimed in claim 6, wherein the unsaturated compound with multi-carboxylic acid groups comprises itaconic acid, maleic acid, maleic anhydride, fumaric acid, methyl maleic acid, methyl fumaric acid, tetrahydrophthalic acid, tetrahydrophthalic anhydride, methyl tetrahydrophthalic anhydride, or glutaric acid.

13. The method of hydrogenating unsaturated compound with multi-carboxylic acid groups as claimed in claim 6, being performed in a batchwise reactor or a continuous reactor.
